(19) United States
(12) Patent Application Publication
(10) Pub. No.: US 2022/0202292 A1
(43) Pub. Date: Jun. 30, 2022

(54) MEASURING SYSTEM

(71) Applicant: ATONARP INC., Tokyo (JP)

(72) Inventors: David ANDERSON, Fremont, CA (US); Prakash Sreedhar MURTHY, Tokyo (JP)

(73) Assignee: ATONARP INC., Tokyo (JP)

(21) Appl. No.: 17/606,877

(22) PCT Filed: Apr. 27, 2020
(86) PCT No.: PCT/JP2020/017886

§ 371 Date: Oct. 27, 2021

Related U.S. Application Data
(60) Provisional application No. 62/840,704, filed on Apr. 30, 2019.

(51) Int. Cl. A61B 5/00; H05B 1/02

(52) U.S. Cl. CPC A61B 5/0035; H05B 1/023; H01S 3/067; A61B 5/0075; A61B 5/0066

(57) ABSTRACT

A system for measurement is provided. The system comprises a core optical module and a scanning interface module. The core optical module is configured to generate a light for generating signals for analyzing an object through the scanning interface module and detect a light including the signals from the object through the scanning interface module. The scanning interface module is changeable for each application and configured to connect with the core optical module by a light transferring unit to scan the object with the transferred light from the core optical module and to receive the light from the object to transfer to the core optical module.

### TECHNICAL FIELD

The invention generally relates to a system for measuring an object.

### BACKGROUND ART

In the publication WO2014/061147, a microscope is disclosed. The microscope includes: a first light dividing part that divides a light flux of light from a light source into a first pump light flux and a second pump light flux; a Stokes light source that receives the second pump light flux as an input and outputs a Stokes light flux: a multiplexing part that multiplexes the first pump light flux and the Stokes light flux to generate a multiplexed light flux; a first light-collecting part that collects the multiplexed light flux in a sample; a first detector that detects a CARS light generated from the sample, the CARS light having a wavelength different from the multiplexed light flux; a second light dividing part that lets at least one of the second pump light flux and the Stokes light flux branch partially as a reference light flux; a second multiplexing part that multiplexes a light flux from the sample and the reference light flux to generate interfering light; and a second detector that detects the interfering light.

### SUMMARY OF INVENTION

One of aspects of this invention is a system comprising a core optical module and a scanning interface module. The core optical module is configured to generate a light for generating signals for analysis by irradiating to an object through the scanning interface module and detect the light including the signals from the target through the scanning interface module. The scanning interface module is changeable for each application and configured to connect with the core optical module by a light transferring unit to scan the object with the transferred light from the core optical module and receive the light from the object to transfer to the core optical module.

In the system of this invention, since the core optical module can be shared by multiple types of scanning interface modules, it is possible to provide systems for multiple applications in a short period of time at low cost. The scanning interface module may be a minimum invasive sampler, a non-invasive sampler, or a flow sampler. The scanning interface module may be a wearable scanning interface, a fingertip scanning interface, a urine sampler, or a dialysis drainage sampler for measuring glucose, hemoglobin A1 c, creatinine, albumin and the like.

### BRIEF DESCRIPTION OF DRAWINGS

The embodiments herein will be better understood from the following detailed description with reference to the drawings, in which:

FIG. 1 shows an embodiment of a system of this invention.

FIGS. 2A and 2B show embodiments of the scanning interface module.

FIG. 3 shows another embodiment of the system.

FIGS. 4A and 4B show an arrangement of an optics plate and a fiber enclosure of an optical core module.

FIG. 5 shows a block diagram of the system.

FIG. 6 shows a block diagram of a fiber laser assembly.

FIG. 7 shows a wavelength plan of the fiber laser assembly.

FIG. 8 shows a wavelength plan of TD-CARS.

FIGS. 9A and 9B show a delay stage.

FIG. 10 shows a block diagram of a temperature control module.

FIG. 11 shows a concept configuration of the optical system of the system.

FIG. 12 shows an example of an arrangement of the optics plate.

### DESCRIPTION OF EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

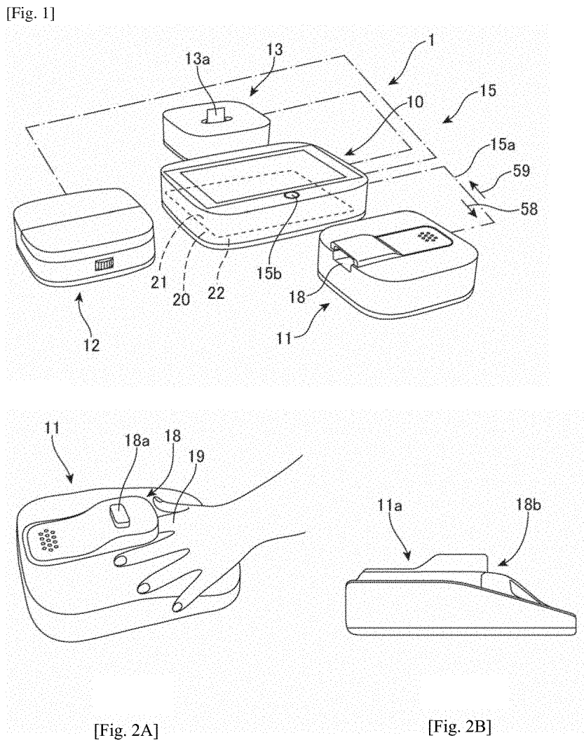

FIG. 1 illustrates a system 1 according to an embodiment of this invention. FIG. 1 shows a core optical module (core module) 10 and a plurality of types of scanning interface modules 11, 12 and 13 for configuring the measuring system 1. For certain applications, a system 1 for measuring the states, composition and others of an object consists of connecting the core optical module 10 and one of scan modules 11 to 13 of either type with a light transferring unit 15. The light transferring unit 15 may be an optical fiber 15a or a free space coupling connector 15b. By using the free space coupling connector 15b, a selected type of scanning interface module among the modules 11 to 13 can be stacked on the core optical module 10. By using the optical fiber 15a, a measuring system 1 can be arranged freely such as stacking, side by side, or keeping the distance between the optical core module 10 and a selected type of scanning interface module among the modules 11 to 13.

One of the systems of an embodiment is a measuring system 1 including the core optical module 10 and a fingertip scanning interface module 11 connected to the core module 10 by the optical fiber 15a. As illustrated in FIG. 2(A), the fingertip type scanning interface module 11 includes an interface 18 for inserting a finger end 19 as an object and a button 18a on the top to put pressure on the finger end to restrict movement at the scanning end. The core optical module 10 is configured to a generate a light 58 for generating signals for analyzing the object 19 through the scanning interface module 11 and detect a light 59 including the signals from the object 19 through the scanning interface module 11. The scanning interface module 11 is changeable for each application and configured to connect with the core optical module 10 by a light transferring unit 15 to scan the object (sample, target) 19 with the transferred light 58 from the core optical module 10 and to receive the light 59 from the object 19 to transfer to the core optical module 10.

In FIG. 1, three different types of scanning interface modules 11, 12, and 13 are shown. Each of the scanning interface modules 11, 12, and 13 is separated from the core optical module 10 but connected with the core optical module 10 via the light transferring unit 15 such as the optical fiber 15a. Types of the scanning interface module are changeable or selectable for each application such as invasive application, non-invasive application, flow measuring application and the like. The basic configuration of all of the types of scanning interface module including the modules 12 and 13 is common with the scanning interface module 11.

The fingertip type scanning interface module 11 is one example of non-invasive samplers. FIG. 2(B) shows a module 11a of another type of non-invasive sampler. The module 11a includes a dome 18b that is similar to a computer mouse for ergonomic positioning of the palm to get the interior information of the living body through the palm using the light from the core optical module 10. A blood glucose monitoring system 1 may be supplied by the core optical system 10 and the non-invasive sampler 11.

The scanning interface module 12 is one example of minimum invasive samplers that may include micro sampling tools such as minimally invasive microneedles and microarrays such that the subject does not feel pain at the time of insertion for sampling body fluids such as subcutaneous tissue fluid. The minimal invasive micro sampling tool is useful for sensing biological information by measuring the concentration of components in body fluids and transdermal administration of drugs. A medication monitoring system 1 may be supplied by the core optical module 10 and the minimum invasive sampler 12.

The scanning interface module 13 is one example of flow samplers that may include a flow path 13a through which a target fluid (object) flows. The target fluid may be urine, dialysis drainage, blood, water, solution, or others. A health management and/or monitoring system 1 may be supplied by the core optical module 10 and the flow sampler 13 as a urine sampler. A dialysis monitoring system 1 may be supplied by the core optical module 10 and the flow sampler 13 as a dialysis drainage sampler.

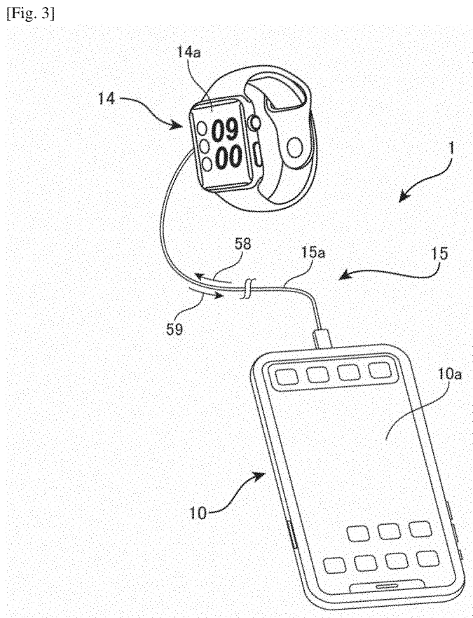

FIG. 3 illustrates a system of another embodiment of this invention. The system 1 includes a wearable scanning interface 14, a portable type optical core module 10, and an optical fiber 15a connecting the wearable scanning interface 14 and the portable type optical core module 10. The wearable scanning interface 14 may be a watch type device or integrated in a watch type communication device such as a smartwatch. In the wearable scanning interface 14, optical elements and/or optical paths for guiding and/or generating light for scanning the object may be provided or integrated in a chip type optical device having sizes of mm order or smaller. The portable type optical core module 10 may have a size of cell phone or integrated in a cell phone or a smartphone. The portable type optical core module 10 may include at least a laser source device, a detector (spectrometer), and a battery, and other optical element may be included in the chip type of optical device installed in the wearable interface 14. The wearable scanning interface 14 may be a pair of glasses-type device such as a smart glass, a pendant type device, an attachment type device, and others. The portable type optical core module 10 may be shared with each type of scanning interfaces that may be changeable. The wearable scanning interface 14 may include a display 14a for outputting measured values by the system 1 and/or other information. The portable core module 10 may include a display 10a for displaying measured values and/or monitoring results by the system 1 and/or other information.

As illustrated in FIG. 1, the core optical module 10 includes an optical bench (optical stand) 20, of which the upper side is an optics plate 21 and the lower side is a fiber laser enclosure 22. On the optics plate 21, a plurality of optical elements constituting optical paths for generating the light 58 are mounted. The fiber laser enclosure 22 is configured to house at least one fiber laser that generates lasers to feed to the optics plate 21. The core optical module 10 includes a stacked structure 20 in which the optics plate 21 and the fiber laser enclosure 22 are stacked. The core optical module 10 may have multiple layered structure, in addition to the optical bench 20, including a power supply board and an electrical control board. The control board may include functions of communication and control of the system, user interface, and power source for the electrical modules and laser modules.

One example of the light 58 for generating signals for analyzing the object 19 is a combination of Raman spectroscopy (RS) and optical coherence tomography (OCT). Both optical imaging and spectroscopy have been applied to the invasive and non-invasive characterization of an object (a target subject). Imaging techniques, such as OCT excel at relaying images of the target subject microstructure while spectroscopic methods, such as CARS (Coherent Anti-Stokes Raman Scattering), can probe the molecular composition of the target subject with excellent specificity.

OCT is a method of obtaining shape information, which reflects a change in the refractive index, using interference between a reflected light from an object (target) and a reference light that has not irradiated the object. CARS is based on a nonlinear optical phenomenon where, when two light beams with different wavelengths are incident on an object, a CARS light that has a wavelength corresponding to the vibration of molecules forming the object is obtained. A plurality of different methods, such as transmissive CARS and reflective CARS, can be arranged regarding the direction of detecting a CARS light to the incident direction of a pump light and a Stokes light.

Time-resolved coherent anti-Stokes Raman scattering or Time-delayed coherent anti-Stokes Rama scattering (TD-CARS) microscopy is also known as a technique for suppressing non-resonant background by utilizing the different temporal responses of virtual electronic transitions and Raman transitions. There is a need for a system that can easily apply such measurement methods to various applications.

The fingertip scanning interface 11, for example, may scan skin of a finger 19 inserted in the interface 18 with the light 58 generated in the optical core module 10 and supplied through the light transferring unit 15, for generating TD-CARS signals and OCT signals, and send the light 59 including signals (lights) of TD-CARS and OCT to the core optical module 10 through the light transferring unit 15. The fingertip scanning interface 11 may be connected by wired or wireless with the core module 10 to communicate with the core module 10 or the cloud through the core module 10.

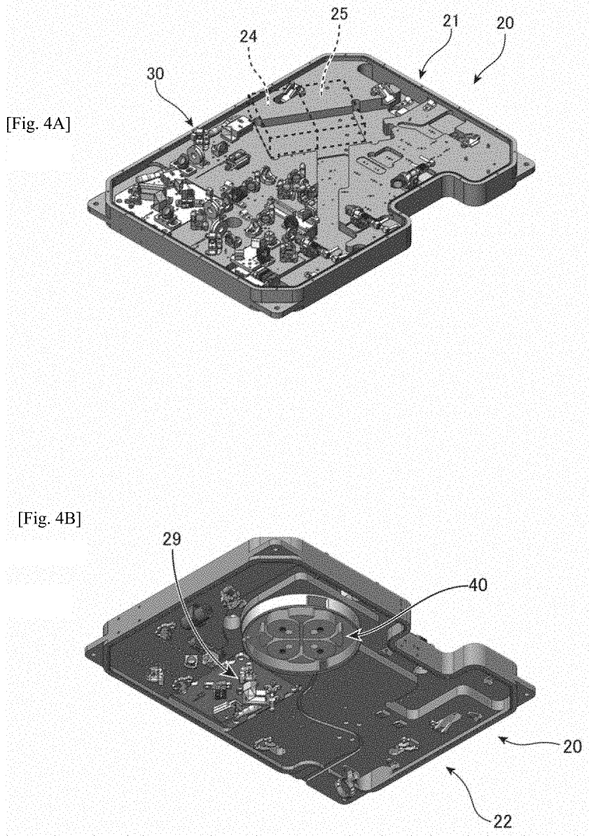

FIG. 4(A) illustrates an arrangement of the optics plate 21 and FIG. 4(B) illustrates an arrangement of the fiber laser enclosure 22. On the optics plate 21, a plurality of optical elements 30 such as mirrors, prisms, dichroic mirrors, and others are mounted for constructing optical paths described hereunder. The optics plate 21 may include a detector 24 for detecting the signals included in the light 59 returned from the scanning interface module 11, and a controller box 25 in which a plurality of modules are housed. On the fiber laser enclosure 22, a fiber laser assembly 40, and a probe delay stage 29 are mounted.

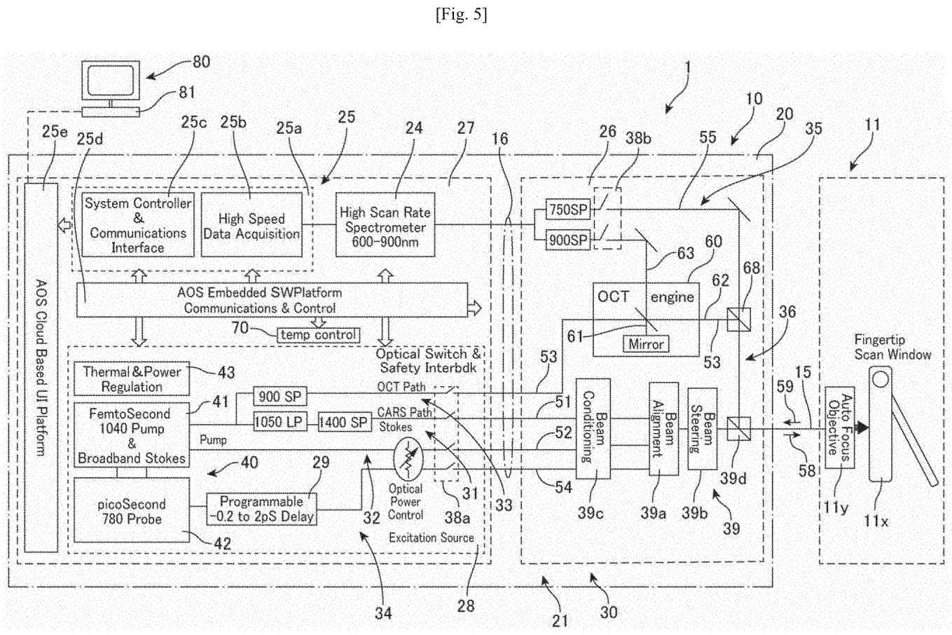

FIG. 5 shows a block diagram of the system 1. The scanning interface module 11 may include a fingertip scan window 11x and an auto focus objective 11y to irradiate (emit) the light 58 from the optical core module 10 to the object and receive the light 59 from the object to transmit to the optical core module 10. The optical core module 10 may include an optical head module 26 and an optical base module 27. The optical head module 26 may be included in the scanning interface module 11, and a connecting 16 between the optical head module 26 and the optical base module 27 may be the light transferring unit. The optical base module 27 includes an excitation source module 28, the detector 24, a temperature control module 70, and the control modules 25a to 25e. The control modules 25a to 25e are housed in the control box 25. The excitation source module 28 includes the fiber laser assembly 40 and the optical paths for supplying light for generating TD-CARS signals and OCT signals. In this fiber laser assembly 40 includes a femto-second fiber laser source module 41 for a Stokes light 51, a pump light 52, and an OCT light 53; a pico-second laser source module 42 for a probe light 54; and a thermal and power regulation module 43 for controlling power supplies to the laser modules 41 and 42.

On the optics plate 21 of the optical bench 20, by using the plurality of optical elements 30 including mirrors, switching elements, reflectors, prisms, lenses, filters such as short wavelength pass filter (SP) and long wavelength pass filter (LP), and others, an optical path 31 for supplying the Stokes light 51 with a first range R1 of wavelengths; an optical path 32 for supplying the pump light 52 with a second range R2 of wavelengths shorter than the first range R1 of wavelengths; an optical path 34 for supplying the probe light 54 with a range of wavelength R4; an optical path 39 for coaxially outputting the Stokes light 51, the pump light 52, and the probe light 54 to the light transmitting unit 15; and an optical path 35 for acquiring the TD-CARS light 55 generated by the Stokes light 51, the pump light 52, and the probe light 54 at the object from the light transmitting unit 15. The TD-CARS light 55 has a range R5 of wavelengths shorter than a range of wavelengths of a CARS light only generated by the Stokes light 51 and the pump light 52. The optical path 34 includes a probe delay stage 29 with an actuator for controlling the emitting of the probe light 54 with the time difference from the emission of the pump light 52.

On the optical plate 21, by using the plurality of optical elements 30, an optical path 33 for supplying the OCT light 53 with a third range R3 of wavelengths shorter than the second wavelength range R2 range of wavelength and at least partly overlapping the wavelength range R5 of the TD-CARS light 55, an optical path 36 for acquiring a reflected OCT light 62 from the light transmitting unit 15, and an OCT engine 60 are also provided. The path 36 includes a dichroic mirror 68 for outputting the OCT light 53 and receiving or returning the reflected light 62 to the OCT engine 60. The OCT engine 60 is configured to split off a reference light 61 from the OCT light 53 and generate an interference light 63 by the reference light 61 and a reflected OCT light 62 through the light transmitting unit 15 from the object. The optical path 39 outputs the OCT light 53 coaxially with the Stokes light 51, the pump light 52, and the probe light 54 to the light transmitting unit 15. The optical path 39 may include a beam conditioning unit 39c, a beam alignment unit 39a, a beam steering unit 39b, and a dichroic mirror device 39d. The dichroic mirror 39d makes the light 58 by combining the light 51, 52, and 54 for generating TD-CARS 55, and the OCT light 53, and separates the returned light 59 that includes TD-CARS light 55 and the reflected light 62. Instead of using the optical elements, or with the use of the optical elements, those optical paths may be provided in or using a chip type optical device. All or a part of those optical paths, instead of providing in the optical core module, may be provided in the scanning module such as wearable model 14.

The core optical module 10 further includes the detector 24 for detecting the TD-CARS light 55 and the interference light 63 of OCT. The detector 24 includes a range of detection wavelengths at least a partially shared with the TD-CARS light 55 and the interference light 63. The core optical module 10 further includes an analyzer 25a for acquiring and analyzing the data from the detector 24. The analyzer 25a may include a high-speed data acquisition module 25b and a system controller and communications interface module 25c. The communications interface module 25c may communicate with the laser assembly 40, the detector 24, the temperature control module 70, switching elements in the optical paths, and other control elements in the core optical module 10 via an embedded switching platform 25d. The core optical module 10 may include a cloud-based UI platform 25e to communicate with the external devices such as a personal computer 80 or server via the Internet. The system 1 including the optical core module 10 and the scanning interface module 11 may communicate with an application 81 installed in the computer 80 to provide a service to a user or users using the system 1.

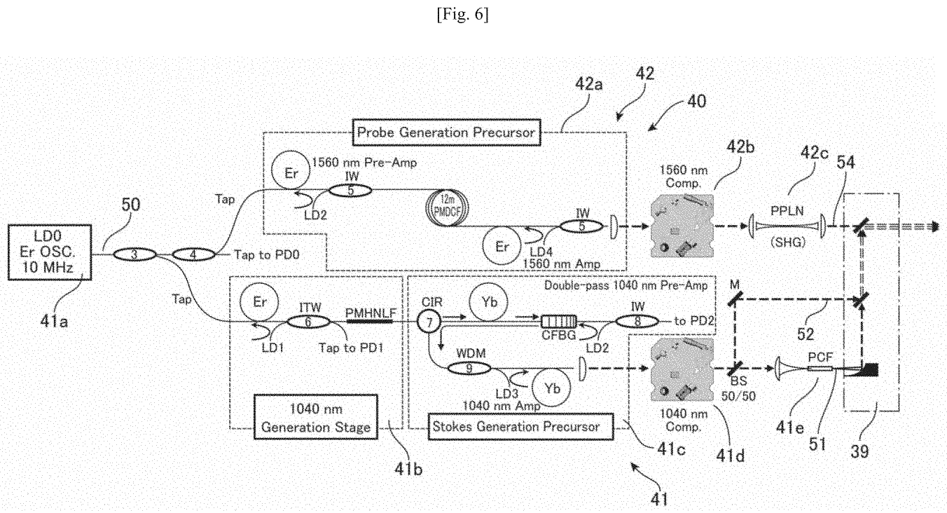

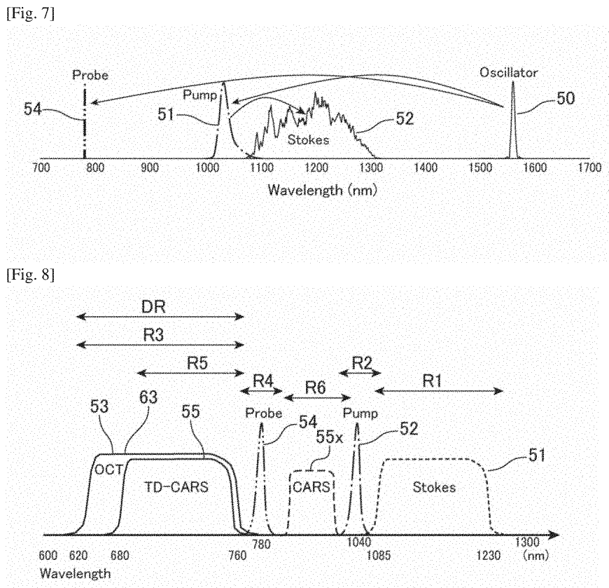

FIG. 6 illustrates one of embodiments of the fiber laser assembly 40. FIG. 7 illustrates a wavelength plan of the fiber laser assembly 40. The assembly may be a MOPA (Master Oscillator Power Amplifier) fiber laser and include a source laser diode LD0 41a to pump Oscillator to produce source laser pulses 50 at 1560 nm. A photo detector PD0 provides feedback signals to ensure that pulses 1560 nm are stable over environment changes. The source laser 50 is split into ports of a probe generation precursor 42a of the pico-second laser source module 42 and a generation stage 41b of the femto-second fiber laser source module 41. In the generation stage 41b, a laser LD1 pumps an Er (Erbium doped) preamplifier spliced to a highly nonlinear fiber (HNLF) to produce 1040 nm to supply to a Stokes generation precursor 41c. In the precursor 41c, a laser LD2 pumps the Yb (Ytterbium doped) preamplifier to amplify 1040 nm pulses, and a laser LD3 pumps Yb high power amplifier to generate 600 mW average power at 1040 nm. A laser outputted from the Stokes generation precursor 41c is supplied to a compressor 41d through a parabolic collimator to generate the Stokes light 51 with a broadband supercontinuum (SC) generated in photonic crystal fiber (PCF) 41e. The laser outputted from the compressor 41d is split to generate the pump light 52.

In the probe generation precursor 42a, a laser LD4 pumps an Er high power amplifier to generate 150 mW average power at 1560 nm. A laser outputted from the probe generation precursor 42a is supplied to a compressor 42b through a parabolic collimator and high power 1560 nm pulses are frequency doubled to 780 nm pulses via PPLN (Periodically Poled Lithium Niobate nonlinear crystal) that acts as SHG (Second Harmonic Generation) to generate the probe light 54. The Stokes light 51, the pump light 52, and the OCT light 53 may include one to several hundred fS (femto second)-order pulses with tens to hundreds of mW. The probe light 54 may include one to several tens pS (pico second)-order pulses with tens to hundreds of mW.

FIG. 7 shows one of the wavelength plans of this optical core module 10. The optical core module 10 should satisfy requirements for several operating modes with minimal hardware and cost. One of the requirements for this optical core module 10 may be that CARS emissions must not overlap TD-CARS emissions. Another one of requirements for this optical core module 10 may be that TD-CARS emissions must overlap OCT excitation for a shared spectrometer range. Yet another one of requirements for this optical core module 10 may be that excitation must have good efficiency through tissue. That is, the Stokes light 51 with the first range R1, the pump light 52 with the second range R2, the probe light 54 with the fourth range R4, and the OCT light 53 and the TD-CARS light 55 with the third range R3 and R5 should be arranged in the range of the optical windows between 600 nm to 1300 nm where the absorbances of major parts of living body such as water, melanin, reduced hemoglobin (Hb), and oxygenated hemoglobin (HbO2) are substantially low.

In the plan shown in FIG. 8, the Stokes light 51 has the first range R1 of wavelengths 1085-1230 nm (400 cm-1˜1500 cm-1), the pump light 52 has the second range R2 of wavelengths 1040 nm, the probe light 54 has the fourth range R4 of the wavelengths 780 nm, OCT light 53 (interference light 63) has the third range R3 of wavelengths 620-780 nm, and TD-CARS light 55 has the range R5 of the wavelengths 680-760 nm. All of the ranges R1, R2, R3, R4 and R5 are included in the range of wavelengths 600 nm to 1300 nm. The second range R2 is shorter than the first range R1, the third range R3 is shorter than the second range R2, the fourth range R4 is shorter than the second range R2 and larger than or included in the third range R3, and the range R5 of TD-CARS 55 is shorter than the fourth range R4 and at least partly overlapping the third range R3. The wavelength range DR of the detector 24 may be 620-780 nm to be shared with TD-CARS 55 and the interference light 63 of OCT. In this plan, only one detector 24 having the detection wavelength range DR shared with the TD-CARS 55 and the OCT light 53 (63) is required. By applying the single and common detector 24 that shares the range DR of detection wavelengths between CARS and OCT detection, the system configuration becomes simplified, and CARS detector fs spectral resolution and OCT imaging depth are increasing. In this optical core module 10, the time-division scan may be required because the CARS light 55 and OCT light 53 (63) use the same spectral range of the single detector 24. Optical switching elements 38a and 38b in the optical core module 10 may be used for time share control.

In this plan, by using the probe light 54 having the shorter wavelength range R4, for example 780 nm, than the range R2 of the pump light 12, the TD-CARS 55 having the wavelength range R5 shorter than the range R4 of the probe light 54 is generated. That is, by using the probe light 54 with the range R4 of wavelengths shorter than the range R6 of wavelengths of the CARS light 55x only generated by the Stokes light 51 and the pump light 52 with a time difference from the emission of the pump light 52, the TD-CARS 55 having the wavelength range R5 shorter than the wavelength range R6 of the CARS light 55x is generated. Accordingly, no interference is made between the TD-CARS 55 and the CARS 55x, and distinct TD-CARS 55 can be detected without interference with the CARS light 55x. The probe light 54 with the range of wavelength shorter than the range R6 of wavelengths of a CARS light 55x only generated by the Stokes light 51 and the pump light 52 may be required to detect a time difference CARS (TD-CARS) 55 that is generated by the Stokes light 51, the pump light 52, and the probe light 54.

Note that the above description does not mean that the CARS light cannot be used as the scanned light 59 to be generated at the object via the scanning module 11, and the scanning light 58 and the scanned light 59 may be for CARS light, SRS (Stimulated Raman Scattering), an infrared light, or any light that may be used as long as it can capture the state of the object as signals and/or spectra. The optical core modules 10 may be a hybrid optical system that includes two detectors for TD-CARS and OCT, or one detector splitting into one half to be used for CARS and the other half used for OCT for detecting the CARS signal and OCT having different spectral ranges.

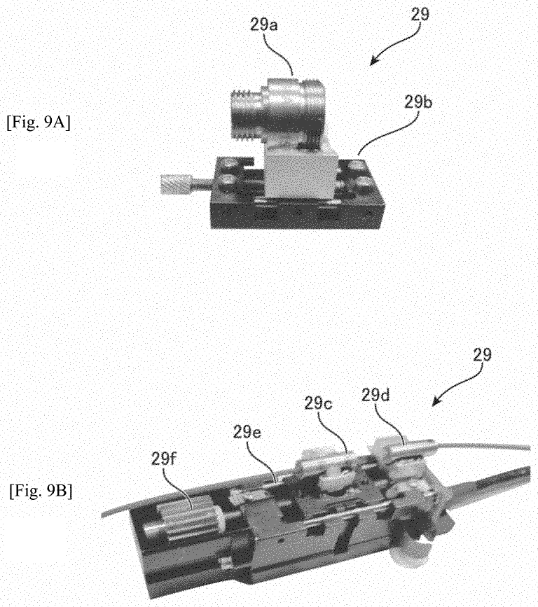

FIG. 9(A) shows an example of a manual delay stage 29 and FIG. 9(B) shows an example of a motorized delay stage 29. Temporal overlap between the probe light 54 and pump/Stokes lights 51 and 52 may be controlled via the manual delay stage (+/−2.5 mm) and/or the motorized delay stage (+/−2.5 mm). In the manual delay stage 29, 1560 nm collimator 29a is mounted on the manual delay table 29b. The motorized delay stage 29 includes a pair of collimates 29c and 29d connected to the optical fibers respectively, a delay table 29e, and a motor 29f. In the motorized optical delay stage 29, the probe light 54 is transferred by the route fiber-in→collimator→free space→collimator→fiber-out. The total travel range may be 10 mm (33 ps).

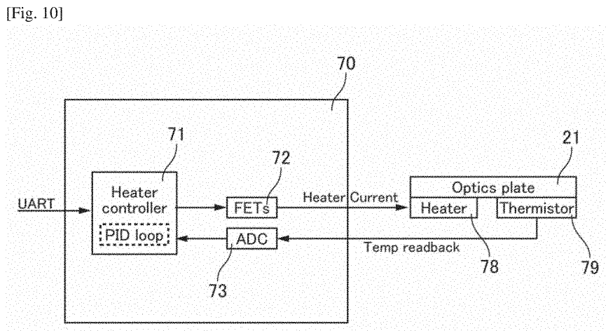

FIG. 10 illustrates the temperature control module 70. In the optics plate 21, since the multiple optical elements 30 are mounted on the optics plate 21 and fine deviations in the position of those elements and/or small changes in the distance between them have a great influence on the optical performance of the optics plate 21, the optics plate 21 and the optical bench 20 shall be rigid, and the temperature of the optics plate 21 shall be constant to avoid the influence of thermal expansion. Accordingly, the core optical module 10 includes the temperature control unit 70 that is configured to control a temperature of the optics plate 21 and/or the optical bench 20.

One example of the temperature control unit 70 includes a heater controller module 71. The heater controller module 71 detects the temperature of the optics plate 21 and/or the environment of the optics plate 21 by a thermistor 79 attached to the optics plate 21, via ADC 73, and control the temperature of the optics plate 21 using a heater 78 via the FETs 72. The heater controller 71 controls the temperature of the optics plate 21 above the ambient temperature to maintain the temperature of the plate 21 at the constant value. The heater 78 may have the heating capacity to maintain the temperature of the plate 21 up to 20C above the averaged ambient temperature such as 25C when the ambient temperature is the lowest such as 15C. The temperature control unit 70 may include a cooling unit such as a Peltier cooling unit. If the optics plate includes an auto tuning unit for compensating the deviations and/or distance changing, the temperature control unit may have a function that avoids the sudden change of the temperature and keeps the temperature gradient in a predetermined range.

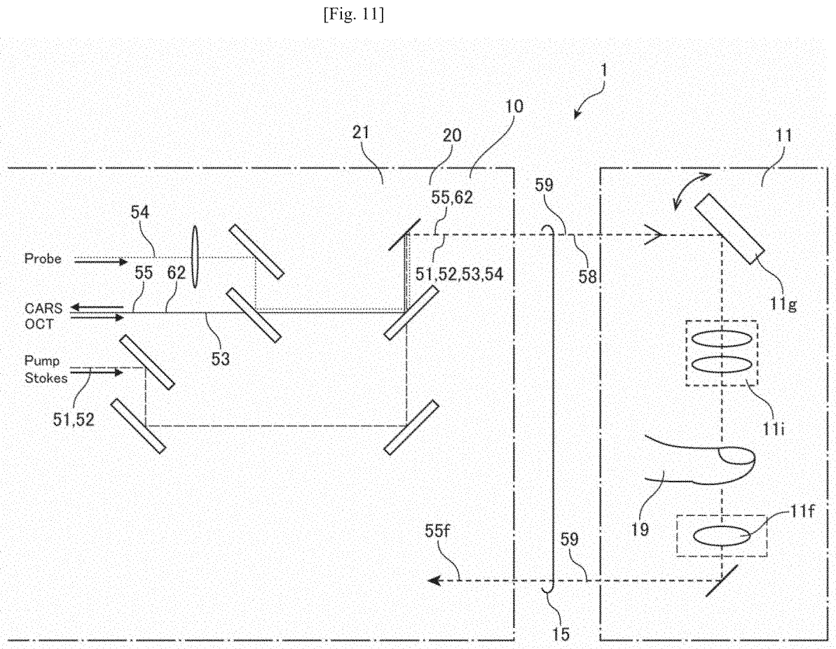

FIG. 11 is a concept configuration between the optical core module 10 and the non-invasive scanning module 11. In the optical core module 10, the Stokes light 51, the pump light 52, and the probe light 54 are combined and delivered to the scanning module 11 as the scan light 58 via the light transferring unit 15 (optical fiber 15a or frees pace coupling 15b). In the scanning module 11, the scan light 58 is irradiated on to the object (target, sample) 19 via a galvanometer 11g and an objective lens module 11i. TD-CARS light 55 is generated by the Stokes light 51, the pump light 52, and the probe light 54 at the object 19, and the backward (Epi) TD-CARS light 55 is returned as the scanned light 59 through the same route as the scanning light 58 to the optical core module 10. The scanning module 11 may include a second objective lens module 11f placed on the opposite side of the object 19 to collect the forward TD-CARS light 55f. The forward TD-CARS light 55f may be returned using the same route of the scanning light 58 as the scanned light 59 via the light transferring route 15.

In the optical core module 10, the OCT light 53 is generated in time division manner for the Stokes light 51, the pump light 52, and the probe light 54 and delivered to the scanning module 11 using the same route of the lights 51, 52, and 54. That is, the OCT light 53 is delivered to the scanning module 11 as the scan light 58 via the light transferring unit 15 (optical fiber 15a or frees pace coupling 15b). In the scanning module 11, the OCT light 53 (scan light 58) shares the same galvanometer 11g and objective lens module 11i and emits to the object (target, sample) 19. The reflected light 62 from the object 19 is returned as the scanned light 59 through the same route as the scanning light 58 to the optical core module 10.

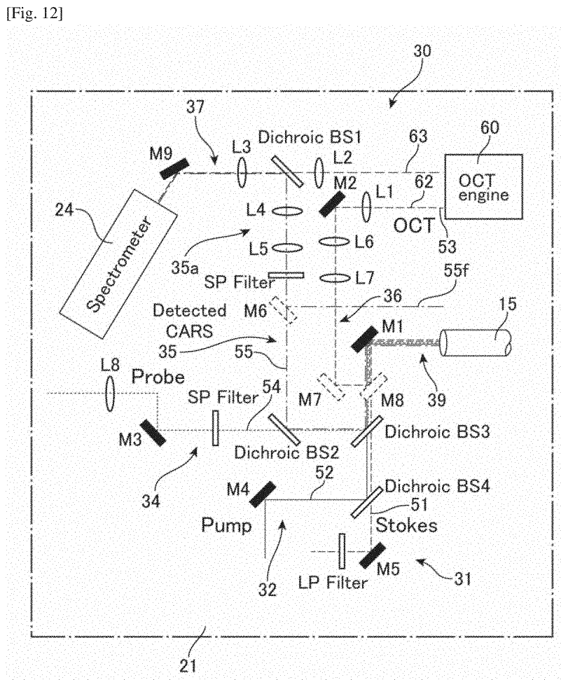

FIG. 12 illustrates one of embodiments of arrangement of the plurality of optical elements 30 on the optics plate 21. A route from the OCT engine 60 to a mirror M1 through a lens L1, a mirror M2, lenses L6 and L7, mirrors M7 and M8 is the optical path 36 for delivering the OCT light 53 onto the object. In this example, the mirrors M7 and M8 are the selection mirrors between OCT light 53 and the returned TD-CARS light 55. When OCT light 53 is engaged, the mirrors M7 and M8 are moved to a pre-set location through a motorized translational stage. The lenses L6 and L7 are the beam expanders that adjust the OCT sample arm beam width to ensure a proper NA to be delivered onto the object. OCT light 53 goes through a galvanometer and a customized multi-element objective, and then is delivered onto the object.

A route from the OCT engine 60 to the detector (spectrometer) 24 through a lens L2, a dichroic beam splitter (dichroic mirror) BS1, a lens L3 and a mirror M9 is a path 37 for the OCT detection. The returned (reflected) OCT light 62 from the target (object) is combined or multiplexed with the reference light 61 to form the interference signal 63 and coupled into the spectrometer 24 through two lenses L2 and L3. In this example, OCT interference signal 63 and CARS light 55 share the same spectrometer 24, which provides the potential to acquire OCT and CARS simultaneously. Time-division between OCT and CARS is however needed if OCT and CARS have overlaps in wavelength. The dichroic beam splitter BS1 is transmissive at the OCT wavelength.

The optical paths 31, 32, and 34 are the paths for delivering the pump light 52, the Stokes light 51, and the probe light 54 onto the target (object sample). In this example, a dichroic beam splitter BS4 combines the pump light 52 and the Stokes light 51, and a dichroic beam splitter BS3 combines the probe light 54 with the pump light 52 and the Stokes light 51. The short pass filter (SP filter) along the probe path 34 filters out the remaining of 1560 nm signal, and the long pass filter (LP filter) along the Stokes path 31 removes the lower wavelength that is out of the region of interest. After the mirror M1, these beams are combined and delivered through the transferring unit 15.

The optical path 35 is the path for the detection of backward CARS (TD-CARS) 55. In this example, a mirror M6 for selecting the forward CARS light 55 collection and the mirrors M7 and M8 for selecting the OCT lights 53 and 63 are moved out of the way through motorized stages. The dichroic beam splitters BS1, BS2 and BS3 reflect the detected CARS signal 55 for collection. The use of the dichroic beam splitter BS1 enables the single spectrometer for both CARS and OCT detection. Lenses L4 and L5 consist of a beam expander to ensure a proper collection NA for spectrometer 24. The short pass filter (SP filter) on this path 35 ensures that only the interested wavelengths are collected by the spectrometer 24.

An optical path 35a that is a part of the path 35 is a route for the detection of forward CARS 55f. In this example, a mirror M6 is moved in place for selecting the forward CARS light 55f collection through a motorized stage. The dichroic beam splitter BS1 reflects the detected CARS signal 55 or 55f for collection. The lenses L4 and L5 consist of a beam expander to ensure a proper collection NA for spectrometer 24. The short pass filter (SP filter) ensures that only the interested wavelengths are collected by the spectrometer 24.

In this system 1, the core optical module 10 and the one kind of the scanning interface module 11 to 14 may be arranged separately, may be stacked, may be arranged in parallel within the distance where the optical fiber can connect the core optical module 10 and the scanning interface module 11 to 14. By providing the highly versatile, common and general purpose core optical module 10, it is possible to easily develop an optimum scanning interface module for each application, that is easy to customize, low in cost, and capable of supplying a system 1 suitable for measurement, research, monitoring and/or self-care in various filed.

In this specification, a system comprising a core optical module and a scanning interface module is disclosed. The core optical module is configured to generate lights for making signals for searching a target and detect the signals from the target. The scanning interface module is separated from the core optical module but connected with the core optical module via an optical fiber or a free space coupling. The scanning interface module is changeable for each application. The scanning interface module is configured to scan the target with the transferred lights from the core optical module for making the signals and to receive the signals from the target to transfer the signals to the core optical module via the optical fiber or the free space coupling. The scanning interface module may be a minimum invasive sampler, a non-invasive sampler, or a flow sampler. The scanning interface module can change for each application such as fingertip scanning and urine scanning for measuring glucose, hemoglobin A1c, creatinine, albumin and the like.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

1-14. (canceled)

15. A system comprising a core optical module and a scanning interface module, wherein
the core optical module is configured to generate a light for generating signals for analyzing an object through the scanning interface module and detect a light including the signals from the object through the scanning interface module; and
the scanning interface module is changeable for each application and configured to connect with the core optical module by a light transferring unit to scan the object with the transferred light from the core optical module and to receive the light from the object to transfer to the core optical module,
wherein the core optical module includes:
an optics plate on which a plurality of optical elements constituting optical paths for generating the light are mounted; and
a temperature control unit that is configured to control a temperature of the optics plate to maintain at a constant value using a heater.

16. The system according to claim 15, wherein the scanning interface module is separated from the core optical module but connected with the light transferring unit.

17. The system according to claim 15, wherein the core optical module includes:
a fiber laser enclosure that is configured to house at least one fiber laser that generates lasers to feed to the optics plate.

18. The system according to claim 17, wherein the core optical module includes a stacked structure in which the optics plate and the fiber laser enclosure are stacked.

19. The system according to claim 15, wherein the temperature control unit controls the temperature of the optics plate above an ambient temperature.

20. The system according to claim 17 wherein the plurality of optical elements include optical elements for:
supplying a Stokes light with a first range of wavelengths and a pump light with a second range of wavelengths shorter than the first range of wavelengths;
supplying a probe light with a range of wavelength shorter than a range of wavelengths of a CARS light generated by the Stokes light and the pump light to emit with a time difference from the emission of the pump light;
coaxially outputting the Stokes light, the pump light, and the probe light to the light transmitting unit; and
acquiring a TD-CARS light generated by the Stokes light, the pump light, and the probe light at the object from the light transmitting unit.

21. The system according to claim 20, wherein the core optical module further includes a probe delay stage with an actuator for controlling the time difference.

22. The system according to claim 20, wherein the plurality of optical elements further include optical elements for:
supplying an OCT light with a third range of wavelengths shorter than the second range of wavelength and at least partly overlapping a range of wavelengths of the TD-CARS light;
coaxially outputting the OCT light with the Stokes light, the pump light, and the probe light to the light transmitting unit; and
acquiring a reflected OCT light from the light transmitting unit,
wherein the core optical module further includes an OCT engine that is configured to split off a reference light from the OCT light and generate an interference light by the reference light and a reflected OCT light from the light transmitting unit.

23. The system according to claim 20, wherein the core optical module further includes a detector to detect the TD-CARS light.

24. The system according to claim 22, the core optical module further includes a detector that includes a range of detection wavelengths, wherein at least a part of the range of detection wavelengths is shared with the TD-CARS light and the interference light.

25. The system according to claim 15, wherein the light transmitting unit includes an optical fiber or a free space coupling.

26. The system according to claim 15, wherein the scanning interface module includes one of a minimum invasive sampler, a non-invasive sampler, and a flow sampler.

27. The system according to claim 15, wherein the scanning interface module includes one of a wearable scanning interface, a fingertip scanning interface, a urine sampler, and a dialysis drainage sampler.
